(19) United States
(12) Patent Application Publication
(10) Pub. No.: US 2014/0074068 A1
(43) Pub. Date: Mar. 13, 2014

(54) MULTI-COMPONENT METHOD FOR REGENERATIVE REPAIR OF WOUNDS IMPLEMENTING PHOTONIC WOUND DEBRIDEMENT AND STEM CELL DEPOSITION

(71) Applicants: Richard Ty Olmstead, Oviedo, FL (US); Joseph Bielitzki, Winter Garden, FL (US)

(72) Inventors: Richard Ty Olmstead, Oviedo, FL (US); Joseph Bielitzki, Winter Garden, FL (US)

(21) Appl. No.: 14/023,971

(22) Filed: Sep. 11, 2013

Related U.S. Application Data
(60) Provisional application No. 61/700,061, filed on Sep. 12, 2012.

(51) Int. Cl. A61M 35/00

(52) U.S. Cl. CPC A61M 35/00
USPC 604/522

(57) ABSTRACT

A new and useful method for regenerative repair of wounds implementing photonic wound debridement and stem cell deposition is presented which consists of a Wound Assessment and Debridement, Wound Infrastructure Building, and Cell Deposition phases. The present invention allows structured regenerative repair of the wound, utilizes robotic systems to inspect, define and debride the wound, and prints an extracellular matrix on the prepared wound site which speeds healing using developing stem cell technologies. This device is believed to be useful in hospitals and clinics, wherein patients with wounds related to skin trauma such as burns, infection, or melanoma are present. An example of a theatre of use is a children's burn ward. This device is also believed to be useful in military hospitals which receive soldiers injured in combat.

### CROSS-REFERENCE TO RELATED APPLICATIONS

This application claims the benefit of U.S. Provisional Application No. 61/700,061, filing date Sep. 12, 2012, and is hereby incorporated by reference in its entirety.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### TECHNICAL FIELD

The present invention is in the technical field of medical devices. More particularly, the present invention is in the technical field of medical devices utilized for wound repair. More particularly, the present invention relates generally to a multi-component method for regenerative repair of wounds implementing photonic wound debridement and stem cell deposition.

### BACKGROUND OF THE INVENTION

Wound treatment is usually conducted using a macro approach that implements global visual assessment, manual preparation of the site, and passive healing mechanisms. In the incident of wounds of larger surface area, a burn for example, the necrotic or otherwise unsalvageable tissues are removed surgically. This removal is often a gross approach which is unable to distinguish the damaged tissue from the immediately adjacent healthy tissue with particulary.

In the applicant's experience there is a deficiency in the existing and prior art wherein the macro approach to wound treatment fails because local anatomy often restricts and dictates the wound treatment method to be utilized. The prior art approach also is deficient because it relies on manual and passive approaches to wound repair, often relying primarily on the body's ability to heal. This ability is often compromised in an injured individual and can take a long time.

In the applicant's experience, there is a need for a multi-component method for regenerative repair of wounds which overcomes the obstacles of the prior art by i) allowing structured regenerative repair of the wound, ii) utilizing robotic systems to inspect the wound, iii) utilizing robotic systems to define the wound, iv) utilizing robotic systems to debride the wound, v) printing an extra-cellular matrix on the prepared wound bed and vi) speeding the healing process by implementing developing stem cell technologies. The method of the present invention is believed to accomplish all of the foregoing objectives.

### SUMMARY OF THE INVENTION

The present invention provides a new and useful multi-component method for regenerative repair of wounds which implements photonic wound debridement and stem cell deposition, allows structured regenerative repair of the wound, utilizes robotic systems to inspect the wound, define and debride the wound, prints an extracellular matrix on the prepared wound site, and speeds healing by implementing developing stem cell technologies. This device is believed to be useful in hospitals and clinics where patients with wounds like burns may present. An example of a theatre of use is a children's burn ward. This device is also believed to be useful in military hospitals which receive soldiers injured in combat.

### I. Overview

### A. Regenerative Repair Method

The present invention accomplishes the noted objectives by implementing a variety of robotic systems. First, the main robot moves a treatment head into the proximity of the wound site. The treatment head then makes a passive assessment of the wound site using a high definition digital camera system integrated with an Optical Coherence Tomographer (OCT). This enables the treating physician or user to inspect the wound both macroscopically and microscopically. The user then develops an initial therapeutic approach according to the findings of the initial assessment.

Next, the robotic system starts interacting with the damaged tissue using a wash system in combination with photonic debridement and sterilization. In advance of and during photonic debridement and sterilization of the wound bed, the system tests for cellular viability using Laser Induced Breakdown Spectroscopy (LIBS). This step of the current method aids the healing process by minimizing the amount of viable tissue removed from the wound bed.

Upom completion of wound debridement and sterilization, Extra Cellular Matrix (ECM) is deposited onto the newly debrided and prepared wound site. Additional structuring, shaping, or annealing of the ECM material is possible using the laser that is also used for photonic debridement. This is accomplished due to inherent properties of the ECM material that allows it to be fixed to the wound bed. The resultant wound bed is then treated by implementing stem cells, antimicrobials, and growth factors to facilitate healing with reduced scarring. The deposition system takes advantage of the structuring and topography of the ECM in the deposition of these constituents.

### B. Debridement

In the current art, there are a variety of techniques are used to debride a wound. These techniques include but are not limited to autolytic, enzymatic, mechanical, surgical, parasitic, and laser debridement. All of these techniques take a unique approach to wound debridement and healing. However, each technique fundamentally falls short of treating the patient in a way that enables rapid healing when used alone. Therefore by combining these current art techniques with regenerative medicine can a new and useful method be created whereby wounds heal 100 quickly, effectively and completely. In the debridement techniques mentioned above, only laser debridement lends itself to rapidly optimizing the wound for the best possible healing.

Laser debridement in the present invention combines several technologies into a common optical head. The main components of the debridement head are the main laser, the LIBS subsystem, a high Definition Camera System and the OCT subsystem. Each of these technologies has been used in medicine for different diagnostic and treatment systems. Depending on the energy source, the interaction of the laser and tissue can be manipulated and suited for various results. Ultrafast variable pulse width, variable frequency lasers (pico-second to femtro-second pulse) have an advantage in that their interaction with tissue can compare to Excimer lasers; However, the frequencies at which they operate are significantly higher.

LIBS has been used in determining characteristics of tissue. LIBS utilizes and compares the known spectral properties and variations between living tissue and damaged or necrotic tissue to determine viability. The OCT system, as it is utilized in the present invention, serves two separate purposes. Firstly, it is used to aid in the visualization of the wound by providing a three dimensional perspective, imagery and topography of the wound prior to treatment. This is similar to work done in the field of ophthalmology where OCT is used to measure the retina. The related laser focal spot placement is also based on OCT measurements. This approach to laser pointing is utilized in ophthalmologic practices to direct three dimensional focal spot placement during cataract surgery.

Next, bio-printing and cell spraying are utilized in the present invention to deposit ECM, antimicrobial agents, cells, and other materials into the targeted wound bed. These methods have been applied in-vivo. The most novel and unprecedented innovation in the current invention is the ability to restructure the wound as necessary to optimize the wound bed for deposition. Development of the bio-printing system leverages methodologies have proven efficacy in the regenerative medicine community. Additionally, the subtracting printing capabilities of the laser debridement system is an enabling technology for ECM deposition and stem cell placement.

Each of these individual subsystems has a proven medical device history. The novel integration of these devices into a common treatment system creates a new, useful and non-obvious treatment method.

### II. Method Overview

The wound treatment method according to the present invention is implemented into a series of phases. The phases are i) Wound Assessment and Debridement, ii) Wound Infrastructure Building and iii) Cell Deposition.

The first phase in wound treatment is defined here as Wound Assessment and Debridement. This phase includes but is not limited to the steps of repair planning, passive wound assessment, wound debridement and viable tissue determination. Upon commencement, the robotic system initially determines the condition of the wound bed by using passive techniques. These techniques allow the user to determine the general condition of the wound. This can be accomplished on site or remotely. Further, attending physicians can consult with other doctors in remote locations by electronically sharing the data obtained by the passive wound assessment portion of the current invention method. This facilitates collaboration and strategizing among professionals prior to commencement of initiating any active treatment of the subject wound.

Once the wound has been analyzed, active debridement of the wound bed begins by utilizing a wash system and scanning laser system. In addition to removing tissue the laser system sterilizes the wound bed during treatment. During debridement, the viability of cells is also tested using Laser Induced Breakdown Spectroscopy (LIBS) to determine when removal of necrotic tissue from the wound bed is complete. Once all of the debris and necrotic tissue is removed, the wound repair moves to Phase II: Wound Infrastructure Building.

Phase II: Wound Infrastructure Building includes but is not limited to the steps of site planning and mapping, configuration optimization and ECM scaffolding deposition.

In this phase, the previously laser-treated wound bed is mapped. This is accomplished in order to assist in making decisions and refining the treatment plan. To that end, additional laser structuring of the wound bed may be performed in order to help optimize the healing process. This additional structuring is termed configuration optimization. Passive wound assessment and wound optimization may be repeated several times as needed throughout the treatment of the wound. After the wound bed is configured, the wound bed infrastructure is built or augmented. Extra cellular matrix (ECM) is deposited in the wound bed in preparation for Phase III: Cell Deposition of the present invention.

ECM scaffolding deposition is achieved by implementing an integrated robotic arm that positions the deposition system proximal to the wound bed. The deposition system is moved along the wound bed while depositing ECM material and building up the infrastructure as indicated by the treatment plan. Upon completion of ECM scaffolding deposition, the wound bed may again be scanned and re-optimized using the main laser system as necessary.

Once the wound bed infrastructure layer is completed, the final phase of the present invention begins. This is known as Phase III: Cell Deposition and includes but is not limited to the steps of site planning and mapping, factor placement, embedded wound care and stem cell placement. During this phase, stem cells, antimicrobial agents, and growth factors may be deposited into the wound bed. The system then repeats the infrastructure and cell deposition steps as necessary.

The phases of the present invention are described in more detail section III. Technical Approach infra.

### III. Technical Approach

### A. Phase I: Wound Assessment and Debridement

The first phase of the present invention is Wound Assessment and Debridement. This phase comprises the general steps of repair planning, wound assessment, wound debridement, and viable tissue determination.

The first step in the first phase of the present system is the development of a treatment plan, or repair planning. The treatment plan utilizes general information collected during the initial medical assessment of the patient prior to being placed in the regenerative repair method according to the present invention.

Once the initial treatment plan is indicated and the repair planning step is complete, the second step of passive wound assessment is initiated. This step entails the use of a vision system working in tandem with a common aperture Optical Coherence Tomographer (OCT). The vision system allows the general condition of the wound to be assessed. The OCT allows the wound to be probed using non-ionizing radiation to determine additional information about the wound. At the completion of the assessment, refinements to the treatment plan are made. Once the assessment of the wound bed is completed, treatment begins. This commences with Wound Debridement.

During Wound Debridement, loose debris is removed from the wound bed by using a wash and drying system. The drying system uses an air knife to remove loose debris. After the wound is initially cleaned, another passive scan is conducted to determine if the wound is ready to be actively treated. Once it is determined that debridement is complete, Viable Tissue Determination commences. The wound bed is then probed using laser induced breakdown spectroscopy (LIBS) to determine cell viability. This process can be used to validate the treatment plan prior to removing larger amounts of necrotic tissue with the main laser. The laser is configurable to remove either small or large amounts of tissue depending on the treatment plan and diagnostic data collected from the wound.

The main laser treats the wound bed by scanning the focal spot of the laser system three dimensionally while ablating tissue. The focal position to set the laser is determined by using topography information obtained from the OCT. After each main laser treatment, the wound assessment and tissue viability process is repeated as necessary in order to fully clean, debride, and prepare the wound for further treatment.

### 1. Passive Wound Assessment

When the patient first interacts with the regenerative repair method according to the present invention, a passive assessment of the wound is conducted. This passive assessment portion consists of two major components—a vision subsystem and an OCT. The vision subsystem can be a camera with imaging optics that are adapted for use with the regenerative repair method according to the present invention. In one embodiment, it is intended to be in a common aperture to the OCT and the main laser, thus a custom lens design would be required to complete the task. Optical lens design codes are used to determine the custom design necessary to achieve high-resolution imaging. The other major component of the passive wound assessment system is the OCT. The OCT is used to interrogate the wound with non-ionizing radiation. Additionally, the OCT is used to direct the positioning of the main laser system by providing data relating to the topography and tomography of the wound bed.

Although the area of the wound bed treated by this method can be any dimension and no limitations of area are claimed herein, in one embodiment of the present invention the OCT scanned a 5 mm×5 mm×2 mm space providing layer information approximately every 25 microns laterally and less than 5 microns axially, thereby providing information beyond what a basic imaging system can provide. Although The OCT is able to image successive layers of the wound bed to determine the relative position to the main laser system focal point. Additionally, the OCT can determine whether tissue damage has propagated outside of the visual wound bed. In an embodiment of the present invention an imaging depth of a few millimeters is sufficient for the tissue debridement subsystem because as the wound is cleaned by the main laser, underlying tissue is gradually exposed. Evidence that a wound is still propagating is sufficient for the tissue debridement system to treat the wound site. A current limitation of the vision and OCT subsystem is that a composite picture of the wound bed will have to be created using successive images collected by the OCT and vision subsystem. This is due to the limited field of view of the optics. However, because of the integration of the vision and OCT systems, the stitching of imagery will be done with a high degree of fidelity. The two components are able to complement each other with the registration of the wound image to image. This allows for a high-resolution image to be made of the entire wound bed. The treating physician or user thereby will have better visualization of the wound because of the integration of technologies.

### 2. Wound Initial Preparation System

Upon the completion of the passive assessment of the wound bed, the initial wound preparation begins. This process engages in order to remove loose debris from the wound. A wash and air knife system are used in concert to remove said debris. An air knife typically engages first and blows an ionized air stream across the wound bed. This removes loose debris from the wound bed. Next a wash is sprayed into the wound bed, followed by a subsequent pass of the air knife. During this pass, the air knife removes excess moisture and any remaining debris from the wound bed. Once the wound bed is clean, washed and dried, active wound assessment can be started.

### 3. Active Wound Assessment

Active wound assessment is accomplished using laser induced breakdown spectroscopy (LIBS). When this laser breaks down the tissue along the wound bed, a plume is formed. By examining the spectral content of the plume, information about the presence or absence of spectral species can be determined. In the case of tissue viability determination, LIBS can be used to determine the state of the tissue. This process requires only a small sample size and does not require prior preparation of the wound bed. In an embodiment of the present invention, LIBS uses pico-grams of material to determine the characteristics of the region, which minimizes the amount of viable tissue that is removed during testing of tissue viability. By using spectral discrimination, a patient's wound can be discreetly treated to ensure that it is clean of dead or necrotic tissue and debris. In the present invention, the LIBS collection optics are outside of the laser scan lens, which allows spectral data to be more efficiently collected by the LIBS system.

### 4. Tissue Removal

The main laser is extremely important to the success of the regenerative repair method according to the present invention. The main laser must be able to treat the wound bed in an efficient manner that allows for optimal healing. A wide variety of laser interactions are possible but for tissue removal the two most useful types of lasers to the present invention are the Excimer and the ultra-fast laser systems. These two types of lasers result in a “clean” interaction with the tissue. “Clean” interaction is defined here as where there is a little to no heat affected zone next to the wound bed. The energy of the Excimer laser's photons directly ionizes electrons, resulting in ablation of the tissue with minimal thermal radiation or conduction. These types of lasers are most commonly used for ophthalmological treatments in industry and have proven to be very effective. One of the main issues with the Excimer laser is that the frequency at which these lasers can be operated is relatively low. For a tissue removal system, use of this type of laser would require that the laser spot be relatively large to ensure that a wound bed could be treated quickly enough to avoid long treatment times for the patient. The larger focal spot also increases the minimum feature size in the wound bed, which may not be ideal. Another type of laser system that can be used to treat with a “clean” interaction is the tunable ultra-fast laser. These lasers remove tissue by using an intensity base method to cause plasma to form. The aspect of the tunable ultra-fast laser that makes it more attractive for use in the present invention is that the laser is capable of working atfrequencies greater than 100 kHz. This allows for a smaller focal spot to be scanned across the wound bed at high frequencies and allows for tissue interaction to be varied according to the pulse width and frequency of the laser. This decreases the minimum feature size possible, as compared to an Excimer system. Additionally, these ultra-fast solid state lasers operate at wavelengths where the optical components will not erode from laser usage. And finally, ultra-fast lasers are based around the use of solid state lasers, whereas Excimer lasers use of poisonous gases such as ArF to lase.

In order to study the feasibility of using an ultrafast laser to treat a wound, a sample of porcine muscle burnt with a soldering iron was treated using a gantry style laser treatment system. For this experiment, the sample was scanned relative to the laser head. The porcine muscle was treated with an ultra-fast laser system. The laser plasma was raster scanned across the wound. As the laser plasma progressed across the sample, it sterilized the wound bed as well as removing a piece of debris from the wound. A color change in the tissue indicated the presence of possibly viable tissue that has been uncovered through laser treatment. In another embodiment of the present invention, the on board LIBS system is used to further determine the viability of the exposed tissue. In another embodiment of the present invention, the imaging system utilizes a lens that corrects for the aberration of the optical head.

### 5. 3D Scanning of Focal Spot

In the laser experiment described supra, the sample was moved relative to the laser focusing optics. This is not practical for the present invention to be used to treat the patient. Instead a 3D scanning of the focal spot is used, which allows the main laser to be scanned across the region of the wound bed being treated without moving the patient. In order for this to be achieved, a defocus module is integrated with a scan system. The scanning system controls the x and y placement of the laser beam using galvanometer mirrors, while the defocus module moves the focused spot in z by adjusting relative lens positions in the optical group inside the defocus module. This allows for an effective treatment volume to be treated. In one embodiment of the present invention, custom optics for the defocus module and a custom telecentric f theta lens are implemented thereby allowing for integration of the vision camera system and OCT optimally into the present invention.

### 6. Debridement System

The debridement system is moved on a robotic arm and placed proximally to the patient.

Because of the design, exact placement of the optical head is not critical. The optical subsystems are designed to manipulate the laser and OCT to place them in the photo-disruption and measurement range of the devices respectively. In addition to the items outlined previously, an embodiment of the present invention includes the addition of a light valve, energy monitor, light ring, and effluent removal system. These additional modules help with the processing of the wound. The light valve will gate and control the magnitude of the intensity of the main laser when the minors of the scanner are positioning to work on a region of wound. The energy monitor is used to measure the laser energy during the procedure and provide a means to control the energy level of the laser if necessary during surgery. The light ring provides general illumination for the camera system. The effluent removal system removes debris that is generated during the treatment of the wound from the plasma treatment of the wound.

During the process of passive wound assessment and tissue removal the axial optical path length of the system is modified by means of a dynamic optical system. This dynamic optical system also allows for lateral translation of the probe beam. In one embodiment of the present invention, this is achieved by the integration of a dynamic defocus module integrated with a galvanometric scanning system. The optical system thereby has the ability to actively adapt to the wound bed through motion of the optical system not the treatment arm. The final focusing optic can be either telecentric or a nominal focusing lens. The use of a telecentric lens allows the field placement of the focal spot to be mathematically mapped (linear to low order polynomial) to the angular deflection of the galvanometric minors. The dynamic motion of the focal position of the system does put special requirements on the reference arm of the OCT system. The reference arm of the OCT must be able to adjust dynamically to match the path length variation of the treatment arm.

The ability to manipulate the focal position is also used by the treatment laser path. The dynamic placement of the focus of the system allows the treatment laser to be adapted and optimized for the removal of tissue or other constituents. In all processes that require that laser system to sculpt the wound bed the mechanical manipulation of the laser enables the precision positioning of the laser with accuracy to the micron level.

The laser system is tunable in energy, pulse width, and frequency. Each of these parameters allow for control of tissue interaction. In one embodiment of the present invention, the regime that is considered is <50 ps to 100 fs. The system of the present invention is thereby able to coagulate, cut and remove tissue using this technique. The Laser also has the ability to form plasma that sterilizes the wound bed during treatment.

### B. Phase II: Wound Infrastructure Building

Phase II of the method according to the present invention is Wound Infrastructure Building. This phase includes but is not limited to the steps of site planning and mapping, configuration optimization, and ECM scaffolding deposition. Wound infrastructure development utilizes many of the modules developed for the debridement subsystem and adds the scaffold deposition system. This approach allows subtractive printing to be possible if there is a need to reshape the wound bed after the deposition of ECM materials.

In an alternate embodiment of the present invention, the wound bed can be treated using the debridement system after the ECM material has been deposited to optimize it for cell deposition and growth.

In an alternate embodiment of the present invention, the laser is selectively gated to make shapes in the ECM material for optimal healing.

Deposition of materials can occur in a plurality of ways—two of which are described here. The first method entails spraying ECM in a liquid form and then structuring the same. The laser system, as described, easily lends itself to this method because any excess ECM can be removed using the laser system. This also allows for standoff placement of the sprayer relative to the wound bed. The second approach is deposition directly onto the surface of the wound using bio-printing. Commercially available bio-printing heads are modified to practice the best method for deposition using a variety of ECM materials. Because of the proposed laser system's ability to subtractive print if necessary, the risks associated with ECM deposition are minimal.

### C. Phase III: Cell Deposition

Phase III of the method according to the present invention is Cell Deposition, and consists of the phases of site planning and mapping, factor placement, embedded wound care and stem cell placement. The cellular deposition portion of this phase is similar to the ECM deposition system of Phase II of the present invention. Similar to the infrastructure building subsystem, the cellular deposition system builds on the work done for both wound debridement and infrastructure building subsystems. The deposition methods developed for ECM application is employed in the cell deposition system. This allows for accurate placement of stem cells, anti-microbial agents, and other factors as they are applied to the wound bed. The methods for deposition will be developed during the development of the infrastructure building subsystem.

The deposition system of the present invention uses controlled dosing of the regenerative medicine constituents including ECM, stem cells, and growth factors by spraying and/or direct deposition techniques. The system uses metered reservoirs to hold the materials like syringes, and vials. The system uses either air or mechanical action to dispense the regenerative constituents. This is important because of the need to maintain sterility patient to patient.

Thus the present invention is believed to provide a new and useful a method for regenerative repair of wounds which allows structured regenerative repair of the wound, utilizes robotic systems to inspect, define, debride the wound, printing an extracellular matrix on the prepared wound site, and speeding healing by implementing developing stem cell technologies. Further features and objectives of the present invention will become apparent form the following detailed description and the accompanying drawings.

### BRIEF DESCRIPTION OF THE DRAWINGS

FIG. 1 is a general flow diagram view of the phases comprising a multi-component method for regenerative repair of wounds implementing photonic wound debridement and stem cell deposition according to the present invention;

FIG. 2 is a detail flow diagram view of Phase I: Wound Assessment and Debridement using various key technologies according to the present invention;

FIG. 3 is a detail flow diagram view of Phase II: Wound Infrastructure Building using various key technologies according to the present invention; and

FIG. 4 is a detail flow diagram view of Phase III: Cell Deposition using various key technologies according to the present invention.

### DETAILED DESCRIPTION OF THE INVENTION

As described above, the present invention provides a new and useful multi-component system for regenerative repair of wounds which implements photonic wound debridement and stem cell deposition, allows structured regenerative repair of the wound, utilizes robotic systems to inspect the wound, define and debride the wound, prints an extracellular matrix on the prepared wound site, and speeds healing by implementing developing stem cell technologies. This device is believed to be useful in hospitals and clinics, wherein patients with wounds related to skin trauma such as burns, infection, or melanoma are present. An example of an theatre of use is a children's burn ward. This device is also believed to be useful in military hospitals which receive soldiers injured in combat. The following description and accompanying drawings disclose at least one version of such a device.

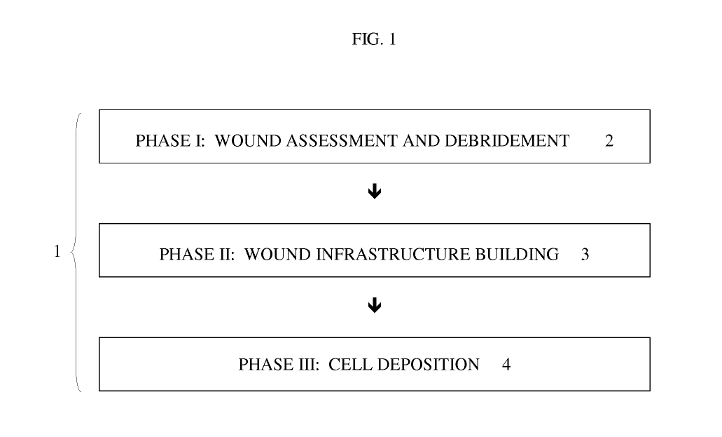

Referring now to the invention in more detail in FIG. 1 there is multi-component method for regenerative repair of wounds implementing photonic wound debridement and stem cell deposition according to the present invention shown generally 1. Said method is comprised of three phases performed sequentially. Said phases comprising phase I: wound assessment and debridement 2, followed by phase II: wound infrastructure building, and followed by phase III: cell deposition 3.

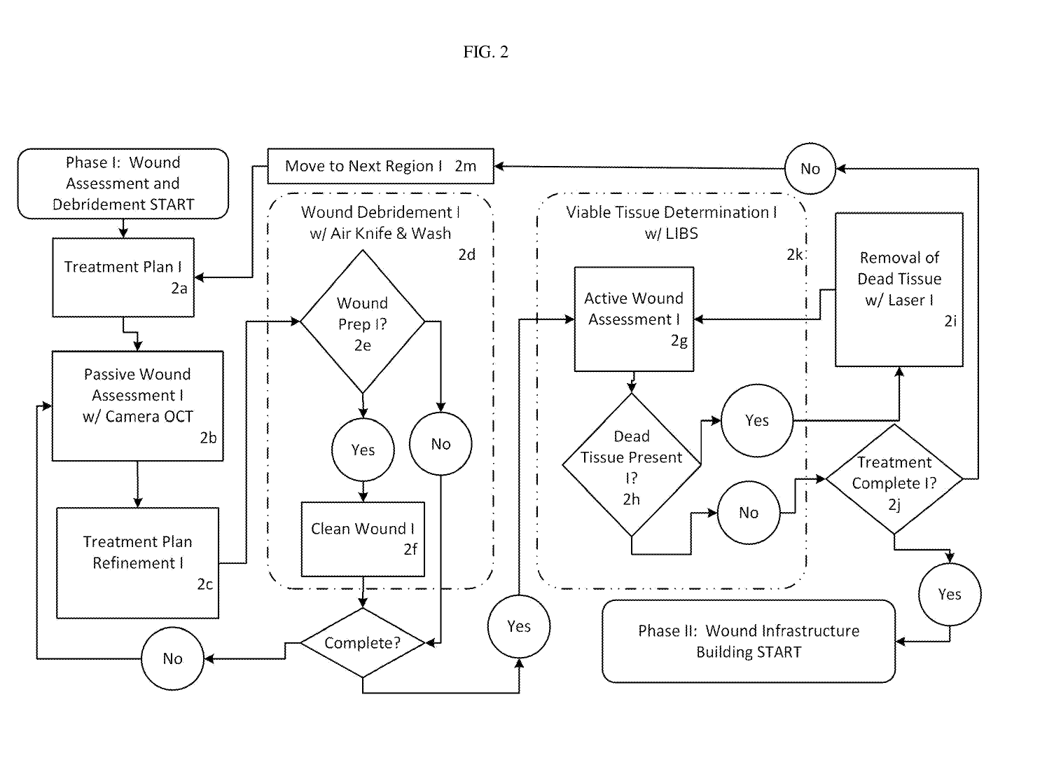

Referring now to the invention in more detail in FIG. 2 there is shown a detailed flow diagram of phase I: wound assessment and debridement 2 of the present invention. Said phase I: wound assessment and debridement 2 is accomplished by an attending medical personnel or user by utilizing steps comprising first formulating a treatment plan I 2a for a target wound bed on a patient. Said treatment plan I 2a is completed utilizing general information collected during the initial medical assessment of the patient prior to being placed in the multi-component method 1 according to the present invention. Next the method continues by performing a passive wound assessment I 2b, where said passive wound assessment I 2b is conducted using a high-definition digital camera system integrated with an Optical Coherence Tomographer (OCT). Following the passive wound assessment I 2b, the user performs treatment plan refinement I 2c according to the findings of the passive wound assessment I 2b.

With a refined treatment plan now developed the user continues the present method by initiating wound debridement I 2d which consists of a wound prep I 2e determination followed by a clean wound step I 2f. Said wound debridement I 2d is conducted to remove loose debris from the wound bed by implementing a wash and dry system, wherein the dry system uses an air knife to remove loose debris from the wound bed.

After completing wound debridement I 2d and determining that no loose debris remains on the wound bed, the user initiates viable tissue determination I 2k. Said viable tissue determination I 2k consists of an active wound assessment I 2g step followed by a dead or necrotic tissue present I 2h determination to determine if dead or necrotic tissue is present at the wound site, and is accomplished using Laser Induced Breakdown Spectroscopy (LIBS) to test for cellular viability.

If dead or necrotic tissue is found the method initiates removal of dead or necrotic tissue I 2i and sterilization of the wound bed using a laser system. Removal of dead or necrotic tissue I 2i is optionally repeated after subsequent active wound assessment I 2g until removal of all dead or necrotic tissue from wound bed is complete.

After completing viable tissue determination I 2k and determining that no dead or necrotic tissue remains, a treatment complete I 2j determination is made to determine successful completion of treatment according to treatment plan I 2a. Once phase I: wound assessment and debridement 2 is complete, the method proceeds to phase II: wound infrastructure building 3.

In more detail, still referring to the invention of FIG. 1, phase I: wound assessment and debridement 2 may further comprise an optional step where the method returns to passive wound assessment I 2b if the wound prep I 2e step is incomplete. Also the method may further comprise optionally returning to passive wound assessment I 2b if the clean wound I 2f step is incomplete. Finally the method may further comprise optionally performing a move to next region I 2m step thereby moving to the next region of the wound bed and repeating phase I: wound assessment and debridement 2 until the treatment plan has been completed as indicated.

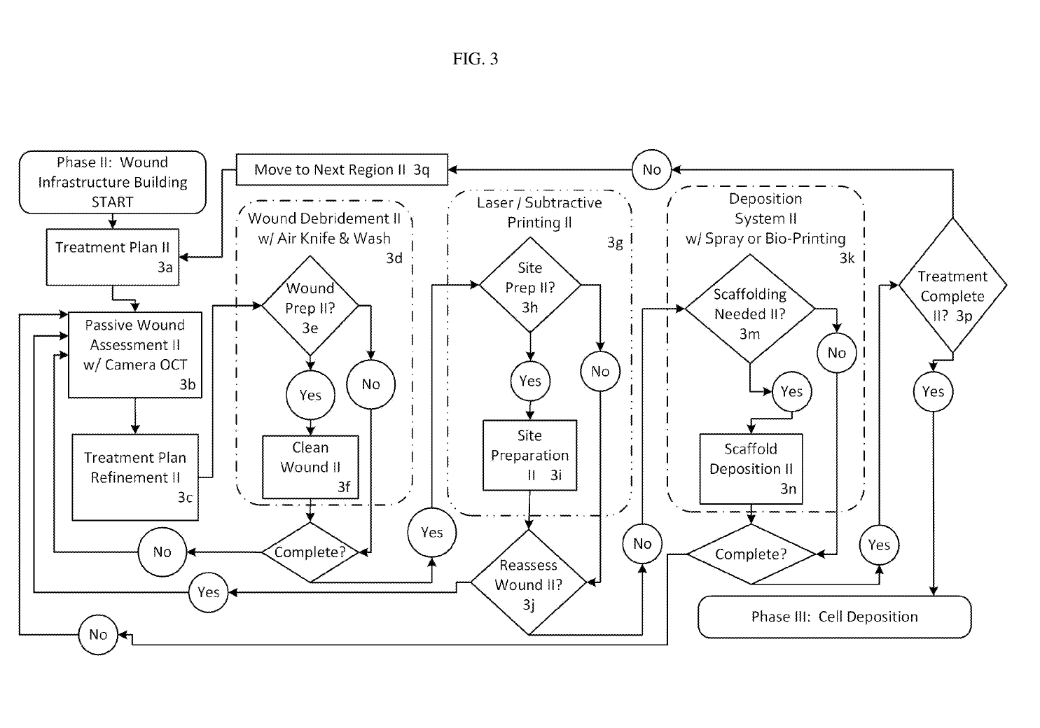

Referring now to the invention in more detail in FIG. 3 there is shown a detailed flow diagram of phase II: wound infrastructure building 3 of the present invention. Said phase II: wound infrastructure building 3 is accomplished utilizing steps comprising first formulating a treatment plan II 3a for the now debrided wound bed on the patient. Treatment plan II 3a utilizes general information collected after completion of phase I 2 of the present invention. Next the user performs passive wound assessment II 3b using a high-definition digital camera system integrated with an Optical Coherence Tomographer (OCT). Based on the finding of the OCT, the user performs treatment plan refinement II 3c.

After refining the treatment plan, the method proceeds towards wound debridement II 3d, which consists of a wound prep II 3e determination followed by a clean wound II 3f step.

Said wound debridement II 3d is conducted to remove loose debris from the wound bed by implementing a wash and dry system, wherein the dry system uses an air knife to remove loose debris from the wound bed.

Next, laser/subtractive printing II 3g is initiated and consists of a site prep II 3h determination followed by a site preparation II 3i step if necessary. Before proceeding, a reassess wound II 3j query is made to determine if further passive wound assessment II should be repeated. If no further passive wound assessment II 3b is needed, the method proceeds by initiating deposition system II 3k. Said deposition system II 3k consists of a scaffolding needed II 3m determination and a scaffold deposition II 3n step. Deposition is accomplished by spraying extra-cellular matrix (ECM) in a liquid form onto the wound bed or deposition directly onto the surface of the wound using bio-printing. After determining if successful completion of treatment according to phase II treatment plan, the method proceeds to phase III: Cell Deposition 4.

In more detail, still referring to the invention of FIG. 3, the site preparation II 3h step can be utilized to reshape the wound bed after the deposition of ECM materials if necessary. Also, the wound bed can be treated using the debridement system after the ECM material has been deposited to optimize it for cell deposition and growth. The laser can be selectively gated to make shapes in the ECM material for optimal healing.

In more detail, still referring to the invention of FIG. 3, phase II: wound infrastructure building 3 may further comprise optionally returning to passive wound assessment II 3b if the wound prep II 3e step is incomplete. Phase II: wound infrastructure building 3 may further comprise optionally returning to passive wound assessment II 3b if the reassess wound II 3j determination yields an affirmative determination.

In more detail, still referring to the invention of FIG. 3, phase II: wound infrastructure building 3 may further comprise optionally returning to passive wound assessment II 3b if the scaffold deposition II 3n step is incomplete. Phase II: wound infrastructure building 3 may further comprise performing a move to next region II 3q step thereby moving to the next region of the wound bed and repeating phase II 3 until the entire wound bed has been treated.

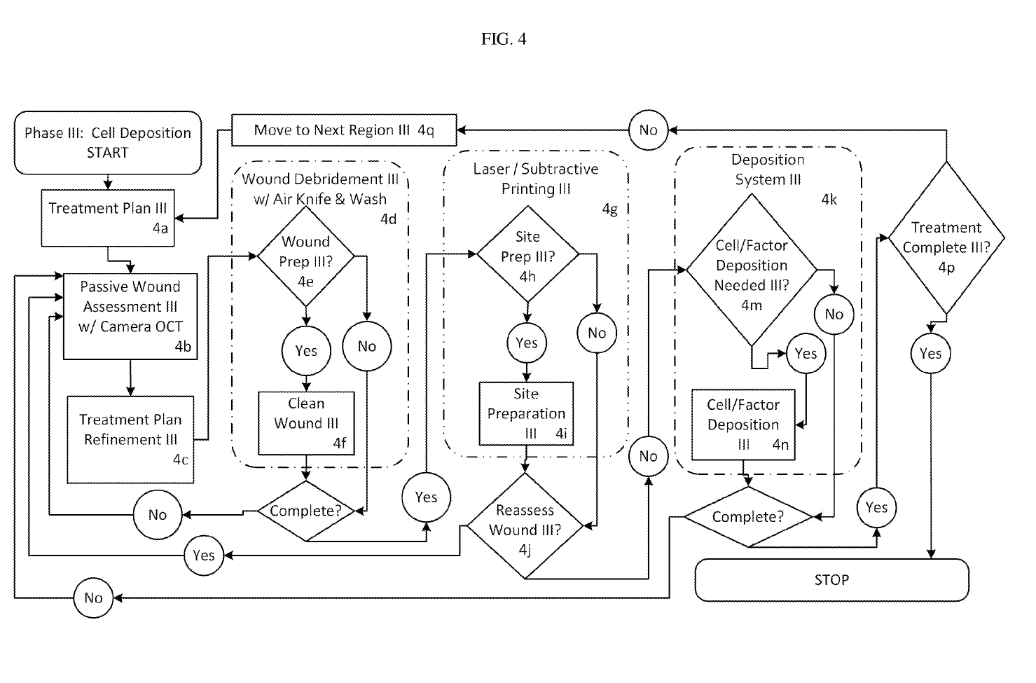

Referring now to the invention in more detail in FIG. 4 there is shown a detailed flow diagram of phase III: cell deposition 4 of the present invention. Said phase III: cell deposition 4 is accomplished utilizing steps comprising first formulating a treatment plan III 4a for the now prepared wound bed with scaffolding on the patient. Treatment plan III 4a is created utilizing general information collected after completion of phase II 3 of the present invention. Next the user performs passive wound assessment III 4b using a high-definition digital camera system integrated with an Optical Coherence Tomographer (OCT). Based on the information collected in the previous passive wound assessment III 4b, treatment plan refinement III 4c is accomplished. Next wound debridement III 4d is initiated and consists of a wound prep III 4e determination followed by a clean wound III 4f step. Wound debridement III 4d is conducted to remove loose debris from the wound bed by implementing a wash and dry system, wherein the dry system uses an air knife to remove loose debris from the wound bed. Once the wound bed 520 debridement is complete laser/subtractive printing III 4g begins. Said laser/subtractive printing III 4g consisting of a site prep III 4h determination followed by a site preparation III 4i step if necessary. Before proceeding, a reassess wound II 3j query is made to determine if further passive wound assessment III 4b should be repeated. If no further passive wound assessment III 4b is needed, the method proceeds by initiating deposition system III 4k. Said deposition system III 4k consists of a cell/factor deposition needed III 4m determination and a cell/factor deposition III 4n step. Said cell/factor deposition III 4n step is accomplished by utilizing controlled dosing of regenerative constituents including ECM, stem cells, and growth factors by spraying and/or direct deposition techniques and utilizing air or mechanical action to dispense the regenerative constituents. Metered reservoirs are used to hold the materials like syringes, and vials.

Finally, after deposition system III 4k is complete according to phase III treatment plan the invention method stops.

In more detail, still referring to the invention of FIG. 4, phase III: cell deposition 4 may further comprise optionally returning to passive wound assessment III 4b if the wound prep III 4e step is incomplete. Also, the method may further comprise optionally returning to passive wound assessment III 4b if the reassess wound III 4j determination yields an affirmative determination. Also the method may further comprise optionally returning to passive wound assessment III 4b if the cell/factor deposition III 4n step is incomplete.

And finally the method may further comprise optionally performing a move to next region III 4q step thereby moving to the next region of the wound bed and repeating phase III 4 until the entire wound bed has been treated.

The previously described versions of the present invention have many advantages, including and without limitation, the properties of allowing structured regenerative repair of the wound, utilizing robotic systems to inspect the wound, iii) utilizing robotic systems to define the wound, iv) utilizing robotic systems to debride the wound, v) printing an extracellular matrix on the prepared wound site, and vi) speeding healing using developing stem cell technologies. The device of the present invention is believed to accomplish all of the foregoing objectives. The invention does not require that all the advantageous features and all the advantages need to be incorporated into every embodiment of the invention.

Although the present invention has been described in considerable detail with reference to certain preferred versions thereof, other versions are possible. Therefore the spirit and scope of the appended claims should not be limited to the description of the preferred versions contained therein.

The reader's attention is directed to all papers and documents which are filed concurrently with this specification and which are open to public inspection with this specification, and the contents of all such papers and documents are incorporated herein by reference.

All the features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. As for “means for” elements, the applicant intends to encompass within the language any structure presently existing or developed in the future that performs the same function. The invention should therefore not be limited by the above described embodiment, method, and examples, but by all embodiments and methods within the scope and spirit of the invention.

What is claimed is:

1. A method for regenerative repair of wounds implementing photonic wound debridement and stem cell deposition comprising:
a) a wound assessment and debridement phase;
b) a wound infrastructure building phase following the wound assessment and debridement phase; and
c) a cell deposition phase following the wound infrastructure building phase.

2. The method in claim 1, wherein the wound assessment and debridement phase is accomplished utilizing steps comprising:
a) formulating a treatment plan I for a target wound bed on a patient, said treatment plan utilizing general information collected during the initial medical assessment of the patient prior to being placed in the regenerative repair system according to the present invention;
b) performing a passive wound assessment I,
said passive wound assessment I is conducted using a high-definition digital camera system integrated with an Optical Coherence Tomographer (OCT);
c) performing treatment plan refinement I according to the findings of the passive wound assessment I;
d) initiating wound debridement I,
said wound debridement I consisting of a wound prep I determination followed by a clean wound step I,
said wound debridement I is conducted to remove loose debris from the wound bed by implementing a wash and dry system, wherein the dry system uses an air knife to remove loose debris from the wound bed;
e) initiating viable tissue determination I,
said viable tissue determination I consisting of an active wound assessment I step followed by a dead or necrotic tissue present I determination to determine if dead or necrotic tissue is present at the wound site,
said viable tissue determination I is accomplished using Laser Induced Breakdown Spectroscopy (LIBS) to test for cellular viability;
f) initiating removal of dead or necrotic tissue I,
said removal of dead or necrotic tissue I using laser system to remove dead or necrotic tissue if found and to sterilize wound bed during treatment,
said removal of dead or necrotic tissue I is optionally repeated after subsequent active wound assessment I until removal of all dead or necrotic tissue from wound bed is complete;
g) performing a treatment complete I determination to determine successful completion of treatment according to phase I treatment plan I; and
h) proceeding to phase II.

3. The method in claim 1, wherein the wound infrastructure building phase is accomplished utilizing steps comprising:
a) formulating a treatment plan II for the now debrided wound bed on the patient, said treatment plan II utilizing general information collected after completion of phase I of the present invention;
b) performing passive wound assessment II,
said passive wound assessment II is conducted using a high-definition digital camera system integrated with an Optical Coherence Tomographer (OCT);
c) performing treatment plan refinement II thereby refining the treatment plan according to the findings of the passive wound assessment II;
d) initiating wound debridement II,
said wound debridement II consisting of a wound prep II determination followed by a clean wound II step,
said wound debridement II is conducted to remove loose debris from the wound bed by implementing a wash and dry system, wherein the dry system uses an air knife to remove loose debris from the wound bed;
e) initiating laser/subtractive printing II,
said laser/subtractive printing II consisting of a site prep II determination followed by a site preparation II step if necessary;
f) making a reassess wound II determination;
g) initiating deposition system II,
said deposition system II consisting of a scaffolding needed II determination and a scaffold deposition II step,
said scaffold deposition II step consisting of spraying extra-cellular matrix (ECM) in a liquid form onto the wound bed or deposition directly onto the surface of the wound using bio-printing;
h) determining successful completion of treatment according to phase II treatment plan; and
i) proceeding to phase III.

4. The method in claim 1, wherein the cell deposition phase is accomplished utilizing steps comprising:
a) formulating a treatment plan III for the now prepared wound bed with scaffolding on the patient,
said treatment plan III utilizing general information collected after completion of phase II of the present invention;
b) performing a passive wound assessment III,
said passive wound assessment III is conducted using a high-definition digital camera system integrated with an Optical Coherence Tomographer (OCT);
c) performing a treatment plan refinement III thereby refining the treatment plan according to the findings of the passive wound assessment III;
d) initiating wound debridement III,
said wound debridement III consisting of a wound prep III determination followed by a clean wound III step,
said wound debridement III is conducted to remove loose debris from the wound bed by implementing a wash and dry system, wherein the dry system uses an air knife to remove loose debris from the wound bed;
e) initiating laser/subtractive printing III,
said laser/subtractive printing III consisting of a site prep III determination followed by a site preparation III step if necessary;
f) making a reassess wound III determination;
g) initiating deposition system III,
said deposition system III consisting of a cell/factor deposition needed III determination and a cell/factor deposition III step,
said cell/factor deposition III step utilizing controlled dosing of regenerative constituents including ECM, stem cells, and growth factors by spraying and/or direct deposition techniques and utilizing air or mechanical action to dispense the regenerative constituents, said cell/factor deposition III step utilizing metered reservoirs to hold the materials like syringes, and vials;
h) determining successful completion of treatment according to phase III treatment plan; and
i) stopping once complete.

5. The method as in claim 2, further comprising optionally returning to passive wound assessment I if the wound prep I step is incomplete.

6. The method as in claim 2, further comprising optionally returning to passive wound assessment I if the clean wound I step is incomplete.

7. The method as in claim 2, further comprising optionally performing a move to next region I step thereby moving to the next region of the wound bed and repeating phase I until the entire wound bed has been treated according to phase I.

8. The method as in claim 3, where the site preparation II step is utilized to reshape the wound bed after the deposition of ECM materials if necessary.

9. The method as in claim 3, where the wound bed is treated using the debridement system after the ECM material has been deposited to optimize it for cell deposition and growth.

10. The method as in claim 3, were the laser is selectively gated to make shapes in the ECM material for optimal healing.

11. The method as in claim 3, further comprising optionally returning to passive wound assessment II if the wound prep II step is incomplete.

12. The method as in claim 3, further comprising optionally returning to passive wound assessment II if the reassess wound II determination yields an affirmative determination.

13. The method as in claim 3, further comprising optionally returning to passive wound assessment II if the scaffold deposition II step is incomplete.

14. The method as in claim 3, further comprising optionally performing a move to next region II step thereby moving to the next region of the wound bed and repeating phase II until the entire wound bed has been treated according to phase II.

15. The method as in claim 4, further comprising optionally returning to passive wound assessment III if the wound prep III step is incomplete.

16. The method as in claim 4, further comprising optionally returning to passive wound assessment if the reassess wound III determination yields an affirmative determination.

17. The method as in claim 4, further comprising optionally returning to passive wound assessment III if the cell/factor deposition step is incomplete.

18. The method as in claim 4, further comprising optionally performing a move to next region III step thereby moving to the next region of the wound bed and repeating phase III until the entire wound bed has been treated according to phase III.
